(19) United States
(12) Patent Application Publication
(10) Pub. No.: US 2004/0229783 A1
(43) Pub. Date: Nov. 18, 2004

(54) Methods of treating intestinal inflammation

(75) Inventors: Charalabos Pothoulakis, Weban, MA (US); Christos Mantzoros, Watertown, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Inc.

(21) Appl. No.: 10/688,194

(22) Filed: Oct. 17, 2003

Related U.S. Application Data
(63) Continuation of application No. PCT/US02/12880, filed on Apr. 22, 2002.
(60) Provisional application No. 60/285,582, filed on Apr. 20, 2001.

(51) Int. Cl. A61K 39/395; A61K 38/17

(52) U.S. Cl. 514/12; 424/143.1

(57) ABSTRACT

Methods for treating intestinal inflammation by inhibiting the activity of leptin or its receptor are described.

### RELATED APPLICATIONS

This application is a continuation of International Application No. PCT/US02/12880, which designated the United States and was filed Apr. 22, 2002, published in English, which claims the benefit of U.S. Provisional Application No. 60/285,582, filed Apr. 20, 2001.

The entire teachings of the above application(s) are incorporated herein by reference.

### BACKGROUND OF THE INVENTION

Leptin is the product of the human homolog of the murine Ob gene (Zhang, Y. et al., 1994. Nature, 372:425-432). Leptin is produced by adipocytes and is distributed through serum to various tissues. Leptin was originally identified because of it role in regulating appetite. This regulation occurs through signaling events centered in the hypothalamus. The binding of leptin to the leptin receptor (Ob-R) triggers the leptin-dependant signaling pathway.

Ob-R exists in several isoforms encoded by the same gene, db (Tartaglia, L. et al., 1995. Cell, 83:1263-1271; Takaya, K. et al., 1996. Biochem. Biophys. Res. Commun., 225:75-83). The Ob-Rb isoform of Ob-R, which contains a long intracytoplasmic domain, has been shown to be important for mediation of leptin's effects. Different forms of Ob-R are generated as splicing variants and Ob-Rb is sometimes referred to as the “long” form of the receptor. Other variants include soluble receptor isoforms and isoforms with shorter intracytoplasmic domains.

Some studies have suggested that leptin and Ob-Rb may be associated with inflammatory conditions. Leptin acting on the hypothalamus regulates neuroendocrine systems important in immune function such as the ACTH-corticosterone axis (Ahima, R. et al., 1996. Nature, 382:250-2; Heiman, M. et al., 1997. Endocrinology, 138:3859-63). More specifically a leptin deficient or leptin resistant state is associated with increased corticosterone levels whereas leptin replacement of leptin deficient rodents results in normalization of corticosterone level. Leptin administration to rats also results in increased levels of the proinflammatory cytokine IL-1β in the rat hypothalamus, and injection of lipopolysaccharide (LPS) stimulates increased leptin mRNA and protein expression. In addition peritoneal macrophages isolated from leptin deficient and leptin resistant mice had decreased phagocytic activity, and expression of proinflammatory cytokines including interleukins and TNFα, and leptin administration to ob/ob mice normalized both responses (Luheshi, G. et al., 1999. Proc. Natl. Acad. Sci. USA, 96:7047-52; Grunfeld, C. et al., 1996. J. Clin. Invest., 97:2152-7; Sarraf, P. et al., 1997. J. Exp. Med., 185:171-5; Loffreda, S. et al., 1998. FASEB J., 12:57-65).

The possibility that leptin may be involved in intestinal inflammation has also been suggested because of leptin's better characterized role in regulating appetite and energy expenditure. However, the functional importance of elevated plasma leptin in the pathophysiology of intestinal inflammation has not been previously examined and the mechanism underlying such a role remains elusive.

### SUMMARY OF THE INVENTION

The present invention is based on the discovery, disclosed herein, of a direct causal link between leptin and intestinal inflammation. As described herein, leptin plays an important role in regulating the severity of enterotoxin-mediated intestinal secretion and inflammation by activating both corticosteroid dependent and independent mechanisms. In particular, it is herein described that during an inflammatory response plasma leptin levels increase and expression of the long isoform of the leptin receptor is increased in intestinal tissue. Also disclosed is evidence that induction of inflammation in a mouse model system is strictly dependent upon the leptin receptor and leptin. Thus, inhibition or modulation of the leptin-mediated inflammatory response, e.g., by inhibiting leptin binding to its receptor, will decrease or completely suppress the leptin-mediated inflammatory response.

The present invention relates to methods of inhibiting or decreasing an inflammatory response in intestinal tissue comprising administering an effective amount of an agent, thereby inhibiting the inflammatory response. Administering the agent can be by means of directly contacting intestinal tissue cells with the agent or by delivering the agent alone or in a composition with an acceptable carrier or delivery vehicle. More specifically, the methods of the present invention encompass methods of inhibiting or decreasing intestinal inflammation in a mammal (e.g., a human) by administering to the mammal an effective amount of an agent such as, for example, a leptin or leptin receptor inhibitor to inhibit or decrease intestinal inflammation. The inflammation may be of the small or the large intestine.

The present invention encompasses methods of treating intestinal inflammation, where the methods comprise inhibiting or modulating leptin activity, leptin binding to the leptin receptor, or the signaling activity of the leptin receptor. The methods disclosed herein contemplate the use of an agent that inhibits, i.e., inhibitors or antagonists, or modulates, e.g., agonists or other effectors, the activity of leptin or the leptin receptor, so that leptin-mediated inflammation is reduced or inhibited. In particular, the use of leptin inhibitors, e.g., small molecules, soluble leptin receptor peptides or fragments, and leptin antibodies; leptin receptor inhibitors, e.g., small molecules, leptin receptor antibodies, leptin analogs and leptin derivatives; and leptin antagonists, e.g., small molecules, antibodies or leptin derivatives. The methods described herein comprise the use of leptin receptor antagonists such as leptin analogs and leptin derivatives (e.g., peptide fragments of leptin that bind specifically to the receptor, but do not induce the inflammatory response, e.g., a signal activity as would normally occur if leptin bound to the receptor). Any combination of leptin inhibitor, leptin receptor inhibitor, leptin antagonist, or leptin receptor antagonist are encompassed by this invention.

Any form of intestinal inflammation can be treated with the methods of the present invention. The inflammation can be mediated by an agent such as a bacterium, a virus or a toxin (e.g., a toxin produced by Clostridium difficile). The inflammation may be associated with a parasitic infection or a disease such as inflammatory bowel disease (IBD), Crohn's disease, ulcerative colitis, acute enterocolitis, autoimmune inflammation or chronic enterocolitis.

In another aspect, the invention features a composition for treating intestinal inflammation. Compositions comprise an agent that inhibits or modulates the activity of leptin or the leptin receptor and a pharmacologically or physiologically compatible carrier. The agent can act as a leptin or leptin receptor inhibitor or agonist. Agents specifically covered by the present invention are the aforementioned leptin inhibitors or modulators, such as leptin antagonists and leptin receptor antagonists. Such agents can be one or more of the following: leptin antibodies, leptin antagonists, non-biologically active leptin analogs (i.e., analogs that bind to the receptor but do not induce an inflammatory response), or leptin receptor antagonists.

As a result of the experiments described herein, which elucidate a proinflammatory role for leptin in the pathophysiology of enteritis and intestinal inflammation, novel methods for treatment of leptin-mediated intestinal inflammation are now available.

### BRIEF DESCRIPTION OF THE DRAWINGS

FIG. 1 is a histogram showing the effect of toxin A in ileal fluid secretion (mg/cm; y-axis) in normal (+/+) and leptin-receptor deficient (db/db) mice (x-axis). Four to eight +/+ and db/db mice were anesthetized and mouse ileal loops were injected with toxin A or buffer. After four hours, mice were sacrificed and ileal fluid secretion was estimated by loop weight to length ratio(mg/cm). Data are means±SEM of 4-8 animals for each group. “*” denotes p<0.001, of treated versus control (buffer-treated) animals for both +/+ and db/db mice; “+” denotes p<0.001 versus TxA treated +/+ mice.

FIG. 2 is a histogram showing the effect of toxin A in ileal fluid secretion (mg/cm; y-axis) in normal (+/+) and leptin deficient (ob/ob) mice (x-axis). Toxin A was injected in ileal loops of +/+ and ob/ob mice or ob/ob mice that have been pretreated with 3 IP injections of leptin (1 μg/gr weight). After four hours, mice were sacrificed and ileal secretion was estimated by loop weight to length ratio (mg/cm). Data are means±SEM of 4-8 animals for each group. * denotes p<0.002 versus buffer-treated mice for both +/+ and ob/ob mice; “+” denotes p<0.007 versus toxin A (TxA)-treated +/+ mice; ** denotes p<0.02 vs TxA-treated ob/ob mice.

FIG. 3 is a histogram showing the effect of toxin A (TxA) in plasma corticosterone levels (μg/dL; y-axis) in normal (+/+) and leptin-deficient (ob/ob) mice (x-axis). Buffer or toxin A was injected in ileal loops of +/+ and ob/ob mice (n=4-6 per group). After two hours, blood was removed from the orbital vein and plasma corticosteroid level was measured with RIA. * denotes p<0.001 versus buffer-injected mice for both +/+ and ob/ob mice; “+” denotes p<0.001 versus buffer-injected ob/ob mice; ** denotes p<0.001 versus TxA-injected +/+ mice.

FIGS. 4A and 4B are histograms showing physiological changes induced in response to toxin A (TxA). FIG. 4A shows reduced TxA-induced ileal fluid secretion in db/db and ob/ob mice. Data are mean±SEM of 5 animals for each group. *, denotes P* 0.01 versus the respective buffer; + denotes P* 0.05 versus TxA of C57BL/6J mice; **, denotes P<0.05 vs TxA of ob/ob mice. FIG. 4B shows increased leptin plasma levels during TxA-induced enteritis. Data are mean±SEM of 5 animals for each group. *, denotes P<0.05 versus the respective buffer or basal leptin levels (after anesthesia, before injection of loops).

FIGS. 5A and 5B are histograms showing corticosterone levels and fluid secretion amounts in genetically modified mice in response to toxin A (TxA). FIG. 5A shows plasma corticosterone levels in db/db mice in response to TxA. Data are means±SEM of 5 loops per experimental condition. * denotes P* 0.001 versus buffer for both C57BL/6J and db/db mice, and + denotes P<0.01 versus buffer injected C57BL/6J; ** denotes P<0.001 versus toxin A-injected C57BL/6J mice. FIG. 5B shows adrenalectomy in db/db mice reverses reduced TxA-mediated fluid secretion. Data are expressed as mean±SEM. *, denotes P<0.01 vs TxA-injected loops of sham adrenalectomized of both genotypes; +, denotes P<0.01 vs TxA-injected adrenalectomized C57BL/6J mice, #, denotes p<0.01 vs TxA injected sham C57BL/6J mice.

FIGS. 6A, 6B, 6C and 6D are a set of four photomicrographs showing the effect of toxin A in leptin receptor (Ob-Rb) expression in mouse ileum of normal (+/+) mice. Ileal loops of normal +/+ mice were injected with either buffer (control) or toxin A. Animals were sacrificed at 15, 30 or 60 minutes, and ileal loops were cut and processed for immunohistochemical detection of Ob-Rb. All sections were examined by confocal microscopy. Results are representative of three experiments per experimental condition.

### DETAILED DESCRIPTION OF THE INVENTION

Leptin is the product of the obese (ob) gene and is secreted by adipose cells (Zhang, Y. et al., 1994. Nature, 372:425-432). Leptin binds to the leptin receptor, Ob-R (Tartaglia, L. et al., 1995, Cell, 83:1263-1271) to induce leptin receptor signaling. The action of leptin to regulate energy balance appears to be primarily through effects in the brain, in particular the hypothalamus. A rising level of leptin, as triglyceride stores increase, is proposed to serve as a negative feedback signal to the brain, resulting in decreased food intake, increased energy expenditure and resistance to obesity. In addition, plasma leptin levels appear to fluctuate in response to intestinal inflammation.

Leptin levels in blood are transiently elevated at the early stages of trinitrobenzene sulfonic acid (TNBS)-mediated colitis in rats and this increase is correlated with the degree of inflammation and anorexia (Barbier, M. et al., 1998. Gut, 43:783-90). Additionally, a study using cholecystokinin B antagonists and a beta3 agonist that decreases leptin secretion showed improvement in the severity of colitis, which in turn could imply that leptin mediates colonic inflammation in the TNBS model of experimental colitis (Barbier, M. et al., 2001. Life Sci., 69:567-80).

Studies with animals lacking either leptin itself (ob/ob) or its receptor (db/db) have shown a requirement for leptin for the control of appetite and development of obesity. Leptin and leptin receptor-deficient mice are hyperphagic, profoundly obese and resistant to insulin (Zhang, Y. et al., 1994. Nature, 372:425-32; Chen, H. et al., 1996. Cell, 84:491-5). The majority of the studies to date related to leptin and the leptin receptor are concerned with the signaling in the hypothalamus that leads to appetite control and energy expenditure. However, apart from the brain, leptin receptors are also expressed in various organs, suggesting that leptin may have different tissue-specific physiologic and pathophysiological effects.

There are only few studies dealing with the effects of leptin in the gastrointestinal tract. Early studies indicated that genes for two different leptin receptor isoforms are present in the intestine (Lee, G. et al., 1996. Nature, 379:632-5; Cioffi, J. et al., 1996. Nat. Med., 2:585-9). Leptin binding to leptin receptors in the rat jejunal mucosa was demonstrated to inhibit absorption of sugars (Lostao, M. et al., 1998. FEBS Lett., 423:302-6). Morton et al. (1998. J. Biol. Chem., 273:26194-26201) also showed the presence of functional Ob-Rb, the “long” isoform of the leptin receptor, on isolated epithelial cells from the mouse duodenum and on the colonic epithelial adenocarcinoma cell line Caco-2. These investigators also discussed the possibility that the presence of leptin receptors in the jejunum may represent an adipo-enteric loop that provides a negative feedback signal from fat stores to the intestine to regulate lipid handling. Another study demonstrates that starvation of mice leads to a 20% decrease in body weight and to a similar decrease in the weight of the intestines (Chaudhary, M. et al., 2000. Digestion, 61:223-9). Starvation also markedly inhibited intestinal epithelial cell proliferation, but leptin administration had little effect on the small intestine and did not stimulate proliferation. Although leptin has been well-characterized for its role in appetite control and energy expenditure, the known association of anorexia and inflammation has led several groups to suggest that leptin may also have a role in the modulation of inflammatory responses.

Since inflammatory bowel disease (IBD), like other inflammatory conditions, is associated with prolonged anorexia, substantial body loss, and delays in linear growth (Kanof, M. et al., 1988. Gastroenterology, 95:1523-7; Kirschner, B. et al., 1990. Acta Pediatr. Scan., 366:98-104), some investigators examined whether leptin may be associated with intestinal inflammation. One study found no differences in the levels of leptin between children and young patients with IBD compared to controls, suggesting that leptin is unlikely to mediate anorexia and growth failure associated with this disease (Hoppin, A. et al., 1998. J. Ped. Gastroenterol. Nutr., 26:500-505). Another study investigated changes in the levels of leptin in several animal models of colonic inflammation (Barbier, M. et al., 1998. Gut, 43:783-90). In the TNBS model of colonic inflammation, elevated plasma leptin concentrations correlated with the degree of inflammation and anorexia during the early stages of intestinal inflammation. Similar leptin overexpression was observed in indomethacin-induced ileitis and in rats with endotoxic shock. However, these changes were transient and no changes in leptin plasma levels were notable at later phases of colonic inflammation. Although this study indicated that leptin may be associated with anorexia observed during colonic inflammation in animals and humans, no functional evidence was presented thus far to indicate that these increased leptin levels mediate anorexia or colonic inflammation in these animal models. Thus, the role of leptin in the pathophysiology of small intestinal or colonic inflammation remains unknown. The present invention is the first demonstration, disclosed herein, of a direct causal link between leptin and inflammation.

In the studies disclosed herein, the role of leptin in fluid secretion and inflammation induced by the TxA from C. difficile, the causative agent of antibiotic associated colitis in animals and humans (Kelly, C. et al., 1994. N. Engl. J. Med., 330:257-62), was examined for the first time. TxA-induced fluid secretion was previously reported to be mediated by proinflammatory cytokines released from neutrophils and other inflammatory cells following exposure of the small intestine and colon to this toxin (Pothoulakis, C. et al., 2001. Am. J. Physiol., 280: G178-G183). It now appears that endogenous corticosteroids also play an important role in fluid secretion, epithelial cell damage and release of proinflammatory cytokines from the intestinal mucosa in response to TxA (Castagliuolo, I. et al., 2001. Am. J. Physiol., 280:G539-G545).

Mice that lack the gene for either leptin or its receptor have reduced fluid secretion following ileal administration of purified TxA in anesthetized animals in vivo. The results presented herein also show that plasma corticosterone in buffer-injected ob/ob mice is higher compared to buffer-injected wild-type mice, consistent with the high basal corticosterone of ob/ob mice. TxA administration increases plasma corticosteroids in both wild-type and ob/ob mice to a similar degree.

Immunohistochemical studies in wild-type mice showed few leptin receptor-positive cells in buffer-exposed ileum and increased expression of these receptors (localized on epithelial and lamina propria cells) after 30 and 60 minutes of TxA exposure. RT-PCR also showed increased leptin receptor mucosal mRNA levels in TxA-exposed ileum of wild-type mice, relative to controls. These data indicate that leptin participates in the pathophysiology of TxA-induced intestinal inflammation, a condition associated with upregulation of its intestinal receptor. Elevated glucocorticoid levels associated with leptin deficiency might be one of the underlying mechanisms for the resistance of ob/ob mice to the development of intestinal inflammation. However, a direct, corticosteroid-independent, proinflammatory role of the leptin receptor during TxA-induced inflammation is also possible based on the data disclosed herein. This is the first demonstration in the literature for a functional role of leptin and its receptor in the pathophysiology of enterotoxin-mediated secretion and inflammation or any other form of inflammation in the gut.

The present invention therefore includes a method of treating leptin-mediated intestinal inflammation, comprising inhibiting or decreasing leptin activity, leptin binding to its receptor, or the signaling activity of the leptin receptor. Such a treatment can be accomplished by administration of an agent. An agent can be any molecule, chemical or biological, that modulates the activity of leptin or the leptin receptor. Administering the agent can be accomplished by directly contacting leptin receptor positive cells with the agent, or by delivery to leptin receptor positive cells of the agent in a composition with a pharmacologically or physiologically acceptable carrier. Methods are known in the art to contact or deliver an agent to a target tissue or tissue-specific cells (e.g., epithelial and lamina propria cells).

The invention encompasses modulation of leptin activity or leptin receptor activity in vertebrates, and, more specifically, mammals. The methods and of the present invention are suitable for veterinary use as well as for treating humans. For example, canines exposed to toxins that result in leptin-mediated intestinal inflammation can be treated using the methods and/or agents described herein.

Leptin-mediated inflammation occurs in intestinal tissues (e.g., the small or large intestine, ileum or colon). This inflammation is characterized by fluid secretion, diarrhea and elevated cytokine levels. The inflammation can be mediated by a bacteria (e.g., Clostridium difficile), a virus or a toxin. Such a toxin can be produced by a bacterium (e.g., TxA produced by C. difficile). Alternatively, the inflammation can be mediated by an autoimmune response (Chan, J. et al., In press. Diabetes). The intestinal inflammation can be that caused by any inflammatory response such as, for example, a parasitic infection, autoimmune inflammation, a response associated with a disease, such as inflammatory bowel disease (IBD), Crohn's disease, ulcerative colitis, acute enterocolitis or chronic enterocolitis.

An agent “modulates” activity if it alters the activity from that which would be exhibited in the absence of the agent. For example, inhibitors decrease activity, e.g., functional inhibitors that interact and block an active site, or competitive inhibitors that compete for binding; antagonists inhibit binding activity, e.g., molecules that reduce binding affinity between a receptor and ligand; and agonists increase binding activity, e.g., molecules that increase binding affinity between a receptor and a ligand. Examples of such molecules include, but are not limited to, leptin antibodies, small molecule agents, leptin agonists, leptin antagonists, non-biologically active leptin analogs, soluble leptin receptors, leptin receptor agonists or leptin receptor antagonists. These agents can be proteins, peptides, peptide analogs, or chemical compounds or derivatives.

The invention encompasses agents that are antibodies and antisera that can be used for inhibiting the activity of leptin and the binding of leptin to its receptor, thereby mitigating the intestinal inflammation. These antibodies can specifically bind to the leptin receptor located on intestinal cells, thus preventing leptin binding to the receptor, and, thereby, inhibiting or decreasing leptin receptor signaling and the resulting leptin-mediated inflammatory response. Such antibodies and antisera can be combined with pharmaceutically-acceptable compositions and carriers to form compositions. The antibodies can be either polyclonal antibodies or monoclonal antibodies.

Leptin, the leptin receptor, or antigenic epitopes of leptin or the leptin receptor can be used to generate antibodies that are specific for leptin or its receptor. For use as an antigen, leptin or the leptin receptor can be recombinantly produced or engineered as described in, e.g., WO 96/05309; U.S. Pat. No. 5,552,522; U.S. Pat. No. 5,552,523; and U.S. Pat. No. 5,552,524, the teachings of which are incorporated by reference. Leptin or the leptin receptor can also be produced by chemical synthesis, or isolated from mammalian plasma using methods well-known to those of skill in the art. For example, leptin used to induce antibody production can be intact protein, e.g., the full-length polypeptide (Zhang, Y. et al., 1994. Nature, 372:425-432).

Specifically included in the present invention are agents that are leptin analogs or derivatives of either leptin or the leptin receptor. Analogs, as used herein, are molecules that are structurally similar to, for example, leptin, and act to compete with leptin for leptin receptor binding sites. Leptin or leptin receptor or derivatives, as used herein, are peptides or proteins having amino acid sequences analogous to endogenous leptin or the leptin receptor. Leptin derivatives can be used, for example, as a competitive inhibitor of leptin binding by competing for leptin receptor binding sites. The present invention includes the use of such leptin derivatives that are able to bind to the leptin receptor, but do not induce the leptin-mediated inflammatory response. Leptin receptor derivatives can be used, for example, to sequester unbound leptin, thereby reducing the leptin levels available to bind and induce endogenous leptin receptors. Analogous amino acid sequences are defined herein to mean amino acid sequences with sufficient identity of amino acid sequence of endogenous leptin to possess the biological activity of endogenous leptin or a slightly altered activity, e.g., reduced leptin receptor binding affinity, as well as analogous proteins that exhibit greater, or lesser activity than endogenous leptin. The derivatives or analogs of the present invention can also be “peptide mimetics,” peptides or proteins that contain chemically modified or non-naturally occurring amino acids. These mimetics can be designed and produced by techniques known to those of skill in the art (see, e.g., U.S. Pat. Nos. 4,612,132; 5,643,873 and 5,654,276, the teachings of which are herein incorporated by reference).

The present invention also encompasses the administration of fusion proteins comprising leptin, leptin receptor, or derivatives thereof, referred to as a first moiety, linked to a second moiety not occurring in the leptin or leptin receptor protein. The second moiety can be a single amino acid, peptide or polypeptide or other organic moiety, such as a carbohydrate, a lipid, or an inorganic molecule. Examples of a second moiety include, for example, maltose or glutathione-S-transferase. The second moiety can also be a targeting moiety used to target the fusion protein to intestinal tissue.

Where the leptin receptor is membrane-bound, the present invention also provides for inhibiting leptin signaling using soluble isoforms of OB-R, e.g., Ob-Re (Takaya, K. et al., 1996. Biochem. Biophys. Res. Commun., 225:75-83) and engineered soluble forms of the leptin receptor. These soluble forms of the leptin receptor would act to bind to unbound leptin, thereby sequestering leptin free in solution and preventing binding of the free leptin to membrane-bound leptin receptor. For these leptin-receptor isoforms and derivatives, part or all of the intracellular and transmembrane domains of the protein are deleted such that the protein is fully secreted from the cell in which it is expressed. The intracellular and transmembrane domains of the leptin receptor can be identified in accordance with known techniques for determination of such domains from sequence information. Commercially and freely available software, such as TopPred2 (Stockholm, Sweden), can be used to predict the location of transmembrane domains in an amino acid sequence, domains which are described by the location of the center of the transmembrane domain, with at least ten transmembrane amino acids on each side of the reported central residue(s).

Systematic substitution of amino acids within the leptin protein can also be used to engineer high-affinity protein agonists and antagonists to the leptin receptor. Accordingly, the engineered leptin would exhibit enhanced or diminished affinity for binding with the leptin receptor. Such agonists and antagonists can be used to suppress or modulate the activity of leptin, thereby mitigating diarrhea or intestinal inflammation. Antagonists to leptin are applied in situations of gut inflammation, to block the inhibitory effects of leptin and mitigate the inflammation.

Candidate leptin receptor inhibitors or antagonists can also be identified by evaluating the binding of leptin to its receptor in the presence and absence of the candidate inhibitor antagonist. Such techniques are well-known to those of skill in the art. Alternatively, candidate leptin receptor inhibitors or antagonists can be identified by measuring leptin receptor signaling activity by the methods described herein (e.g., measurement of fluid secretion).

Administering agents of the present invention can be accomplished either by administering the agent alone (naked administration) or by administering the agent as part of a composition. Modes of administering the agents or compositions of the present inventions include aerosol, ingestation, intravenous, intramuscular, intraperitoneal, intrastemal, subcutaneous and intraarticular injection and infusion.

The formulations include those suitable for oral, rectal, nasal, topical (including buccal and sublingual), intrauterine, vaginal or parenteral (including subcutaneous, intraperitoneal, intramuscular, intravenous, intradermal, and epidural) administration. The formulations may conveniently be presented in unit dosage of therapeutically effective amounts and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

The compositions containing inhibitors of leptin or the leptin receptor may also contain other proteins or chemical compounds. The composition may further contain other agents which either enhance the activity of the inhibitor or compliment its activity or use in treatment. Such additional factors and/or agents may be included in the composition to produce a synergistic effect with the inhibitor of leptin or the leptin receptor, or to minimize side effects. Pharmaceutical or physiological compositions for parenteral injection comprise pharmaceutically or physiologically acceptable, herein used interchangeably, sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by trapping the drug in liposomes or microemulsions that are compatible with body tissues. Additionally, administration of the inhibitor of leptin or the leptin receptor of the present invention may be administered concurrently with other therapies.

Alternatively, it may be undesirable to administer the protein systemically because of side-affects. To eliminate pleiotropic effects of administering an agent included in the present invention, it would be useful to deliver (or target) the agent to a specific tissue (e.g., intestinal tissue or leptin receptor positive epithelial or lamina propria cells). One way to deliver the agent to a specific tissue is to conjugate the protein with a targeting agent. For example, the protein can comprise a peptide to target the leptin receptor to a specific tissue or cell type, e.g., intestinal tissue or cells. Such targeting molecules are well known to those of skill in the art.

Agents can be used in compositions with carriers known in the art. Such carriers can be used as vehicles that target specific tissues or cell types (e.g., intestinal tissue or leptin receptor positive epithelial or lamina propria cells), are they can be used to increase the stability or efficacy of the agent. Such a composition can also contain (in addition to inhibitor and a carrier) diluents, fillers, salts, buffers, stabilizers, solubilizers, and other materials well known in the art. The term “pharmaceutically acceptable” can be used interchangeably with “physiologically acceptable” to mean a non-toxic material that does not interfere with the effectiveness of the biological activity of the active ingredient(s). The characteristics of the carrier will depend on the route of administration. In addition, an agent, e.g., inhibitor of leptin or the leptin receptor, may be active as a monomer or multimer (e.g., heterodimers or homodimers) or may complex with itself or other proteins or molecules. As a result, compositions of the invention may comprise an agent in such multimeric or complexed form. Such multimers, or complexes, are especially useful, for example, to prolong the half-life of the protein in circulation.

The agents of the present invention can be in the form of a liposome in which the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

The compositions can be administered intravenously, as by injection of a unit dose, for example. The term “unit dose” is an effective amount of the agent that, when used in reference to a composition of the present invention, refers to physically discrete units suitable as unitary dosage for the subject, each unit containing a predetermined quantity of active material calculated to produce the desired effect in association with the required diluent, i.e., carrier or vehicle. As used herein, an effective amount of an agent is that determined by one of ordinary skill in to be the amount necessary to decrease or completely inhibit the inflammatory response mediated by leptin and the leptin receptor in a specific tissue or cell. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions that may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

When an effective amount of the inhibitor of leptin or the leptin receptor of the present invention is administered orally, the composition of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% inhibitor of the present invention, and preferably from about 25 to 90% inhibitor of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of inhibitor of the present invention, and preferably from about 1 to 50% inhibitor of the present invention.

When an effective amount of the inhibitor of leptin or the leptin receptor of the present invention is administered by intravenous, cutaneous or subcutaneous injection, inhibitor of the present invention will be in the form of a pyrogen-free, parenterally acceptable aqueous solution. The preparation of such parenterally acceptable inhibitor solutions, having due regard to pH, isotonicity, stability, and the like, is within the skill in the art. A preferred pharmaceutical composition for intravenous, cutaneous, or subcutaneous injection should contain, in addition to the inhibitor or agonist of the present invention, an isotonic vehicle such as sodium chloride, Ringer's solution, dextrose, dextrose and sodium chloride, lactated Ringer's solution, or other vehicles known in the art. The pharmaceutical composition of the present invention may also contain stabilizers, preservatives, buffers, antioxidants, or other additives known to those of skill in the art.

By “contacting” is meant not only topical application, but also those modes of delivery that introduce the composition into the tissues, or into the cells of the tissues (e.g., intestinal tissue or leptin receptor positive epithelial or lamina propria cells).

Use of timed release or sustained release delivery systems are also included in the invention. Such systems are highly desirable in situations where surgery is difficult or impossible, e.g., patients debilitated by age or the disease course itself, or where the risk-benefit analysis dictates control over cure.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

Additionally, osmotic minipumps may also be used to provide controlled delivery of high concentrations of inhibitor or agonist of leptin or the leptin receptor through cannulae to the site of interest (e.g., delivery of the inhibitor specifically to, for example, intestinal tissue or leptin receptor positive epithelial or lamina propria cells). The biodegradable polymers and their use are known to those of skill in the art, for example, as detailed in Brem et al. (1991. J. Neurosurg. 74:441-446), which is hereby incorporated by reference in its entirety.

The methods of the present invention contemplate single as well as multiple administrations, given either simultaneously or over an extended period of time. In addition, agents suitable for use in the present invention can be administered in conjunction with other forms of therapy, e.g., immunotherapy. The duration of intravenous therapy using the pharmaceutical composition of the present invention will vary depending on the severity of the disease being treated and the condition and potential idiosyncratic response of each individual recipient. It is contemplated that the duration of each application of the inhibitor of the present invention will be in the range of 12 to 24 hours of continuous intravenous administration. Ultimately the attending physician will decide on the appropriate duration of intravenous therapy using the pharmaceutical composition of the present invention.

Preferred unit dosage formulations are those containing a daily dose or unit, daily sub-dose, or an appropriate fraction thereof, of the administered ingredient. It should be understood that in addition to the ingredients, particularly mentioned above, the formulations of the present invention may include other agents conventional in the art having regard to the type of formulation in question.

This invention is illustrated further by the following examples, which are not to be construed as limiting in any way.

### EXAMPLES

### Example 1

### Reduced TxA-Induced Secretion in db/db Mice

Twelve-week-old male wild-type (+/+) and leptin-receptor deficient (db/db) mice weighing 20-25 g were housed under controlled conditions on a 12-12 hour light dark circle. Mice were fasted for 16 hours and then anesthetized with a mixture of ketamine (0.9 mL) and xylazine (0.1 mL) in 9 mL of sterile water at a dose of 0.15 mL per 20 grams of body weight. A laparotomy was then performed and two 3-5 cm-long loops were formed at the terminal ileum as previously described (Pothoulakis, C. et al., 1994. Proc. Natl. Acad. Sci. USA, 91:947-51; Castagliuolo, I. et al., 1999. J. Clin. Invest., 103:843-849). Loops were then injected with either 0.4 mL of phosphate buffer saline (PBS) (pH 7.4) containing 10 μg of purified TxA or buffer alone (control). The abdomen was then closed and animals were placed on a heating pad at 37° C. for the duration of the experiment. After 4 hours, animals were sacrificed with CO₂ inhalation and fluid secretion was estimated as the loop weight to length ratio as previously described (Pothoulakis, C. et al., 1994. Proc. Natl. Acad. Sci. USA, 91:947-51; Castagliuolo, I. et al., 1999. J. Clin. Invest., 103:843-849).

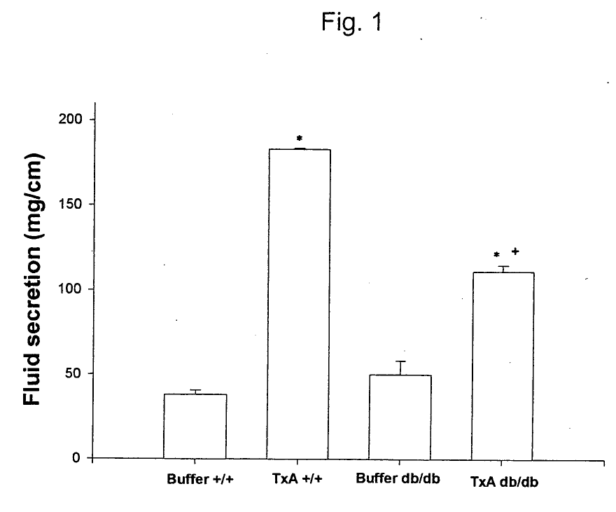

As can be see in FIG. 1, basal fluid secretion in response to buffer injection was comparable between wild type and db/db mice. In wild-type mice, TxA caused a 4.7-fold increase in fluid secretion compared to buffer-injected loops (FIG. 1). However, in db/db mice, TxA increased fluid secretion only by 2.2-fold which is significantly lower (by 39.9%) compared to fluid secretion obtained in db/db mice following TxA exposure (FIG. 1).

### Example 2

### Reduced TxA-Induced Secretion in ob/ob Mice

The effect of leptin administration was observed by comparing the effect of TxA on ileal fluid secretion in normal +/+ mice (wild-type) and mice deficient in leptin itself. In these experiments, twelve week-old male wild-type and leptin-deficient (ob/ob) mice weighing 20-25 g were housed and anesthetized as described above. A laparotomy was performed two, 3-5 cm-long loops were formed at the terminal ileum as described above, and injected with either 0.4 mL of phosphate buffered saline (PBS) (pH 7.4) containing 10 μg of purified TxA or buffer alone (control). In some experiments, 3 single IP (a mouse recombinant from Sigma) doses of leptin were injected (1 μg per gram of body weight) i.p. to ob/ob mice (n=4-8) 20, 14, and 0.5 hr prior to TxA administration. The abdomen was then closed and animals were placed on a heating pad at 37° C. for the duration of the experiment. After 4 hours animals were sacrificed with CO₂ inhalation and fluid secretion was estimated as the loop weight to length ratio as previously described (see Example 1).

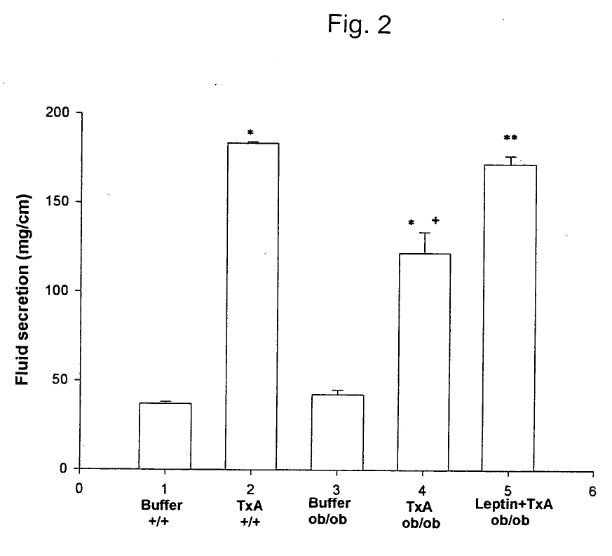

Our results showed that TxA induced a 4.9-fold increase in fluid secretion in wild-type mice compared to injection of buffer (FIG. 2). However, in ob/ob mice TxA induced only a 2.8-fold increase in secretion compared to buffer treatment, which is significantly lower (by 33.46%) compared to TxA-induced secretion in wild-type mice (FIG. 2). In addition, pretreatment of ob/ob animals with leptin resulted in a TxA-induced secretion statistically indistinguishable from the secretion seen in normal, wild-type mice in response to TxA (FIG. 2). Taken together the results shown in FIGS. 1 and 2 and previous data demonstrating that the secretory effects of TxA are mediated by release of proinflammatory cytokines (Pothoulakis. C. et al., 2001. Am. J. Physiol., 280:G1178-G1183.), demonstrate that leptin and its receptor mediate fluid secretion and inflammation during TxA enteritis.

### Example 3

### Increased Corticosteroid Levels in ob/ob Mice before and after TxA Administration

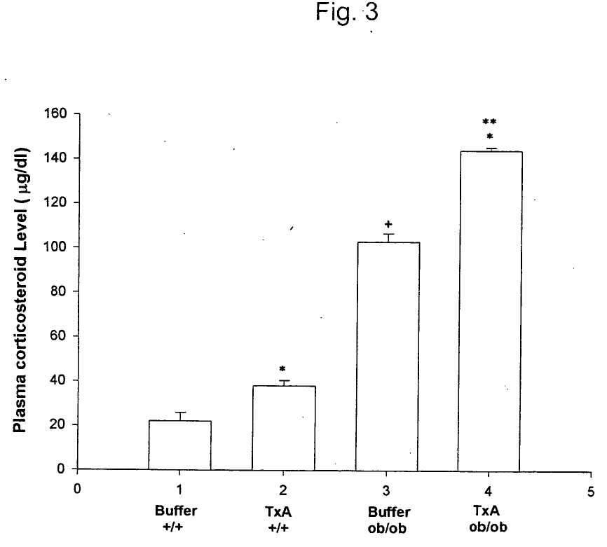

Because of the known association between leptin and the hypothalamic-pituitary-adrenal axis, corticosteroid levels before and after intraluminal TxA administration was compared between wild-type and ob/ob mice. Blood samples (0.2 mL) were collected from the retro-orbital plexus either after anesthesia or 2 hrs after TxA or buffer injection. Blood samples were centrifuged (800 g×10 minutes at 4° C.), plasma was collected and aliquots were stored at −80° C. Corticosterone levels were measured by a radioimmunoassay kit (ICN Biomedicals, Inc., Costa Mesa, Calif., USA as previously described (Castagliuolo, I. et al., 2001. Am. J. Physiol., 280:G539-G545). Our results showed that plasma corticosterone in buffer-injected ob/ob mice was 4.6-fold higher compared to buffer-injected wild type mice, consistent with the high basal corticosterone of ob/ob mice (FIG. 3). Moreover, TxA administration increased plasma corticosteroids in both wild-type and ob/ob mice to a similar degree (1.74-, and 1.40-fold, respectively, FIG. 3). Since previous results indicated that endogenous cortico steroids are important in the modulation of TxA-mediated secretion and inflammation, the results shown in FIG. 3 indicate that the effects of leptin in TxA-induced secretion may be mediated, at least in part, by altered secretion of cortico steroids.

### Example 4

### TxA Increases Intestinal Leptin Receptor mRNA Levels in Normal Mice

Since the results shown in FIG. 1 indicated the importance of the leptin receptor in the mediation of TxA-elicited fluid secretion, the levels of expression of the leptin receptor was examined by RT-PCR. In these experiments, either TxA or buffer was injected into loops of terminal ileum (see Examples 1 and 2, above) of wild-type mice. After 2 hours, animals were sacrificed and the colonic tissue were processed for isolation of total RNA. Total RNA was isolated by guanidium thiocyanatephenol-phenol-chloroform extraction. RNA (1 μg) was reversed transcribed using random hexamer primers (0.1 μg), deoxyribonucleoside triphosphates (5 mM), 5×RT buffer, RNasin (40 U), and Moloney murine leukemia virus RT (200 U) in a total volume of 25 μL (all reagents from Promega, Madison, Wis., USA). The mixture was incubated at 37° C. for 60 minutes and the resulting complementary DNA (cDNA) was stored at −20° C. The following primers were used for amplifying a 500 base-pair fragment corresponding to part of the extra cellular domain of the leptin receptor (amino acids 293-460): primer Ob-Rall (500 bp) upstream:5′-ACA GCG TGC TTC CTG GGT CTT C-3′ (SEQ. ID. NO: 1) and Ob-Rb (downstream):5′-TGG ATA AAC CCT TGC TCT TCA-3′ (SEQ. ID. NO: 2). All PCR amplifications were performed using 40 cycles using a 60 s denaturation step at 94° C., a 60 s annealing step at 55° C., and a 90 s extension step at 72° C. Ten μL of the product was loaded onto an agarose gel and the fluorescence of the ethidium bromide stained band was recorded. TxA-stimulated increased leptin receptor mucosal mRNA levels as compared to control levels. Statistical analysis of the results indicated a 42.2%, (p<0.001) increase in leptin receptor mRNA compared to controls (Wlk, M. et al., 2001; Gastroenterology, In press).

### Example 5

TxA was purified from culture supernatants of C. difficile strain 10,463 as previously described (Pothoulakis, C. et al., 1991. J. Clin. Invest., 88:119-25). Protein concentrations were determined by the bicinchoninic acid assay method (Pierce Laboratories, Rockford, Ill.).

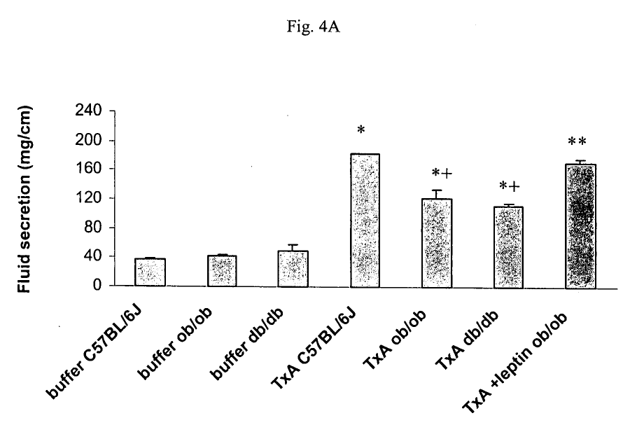

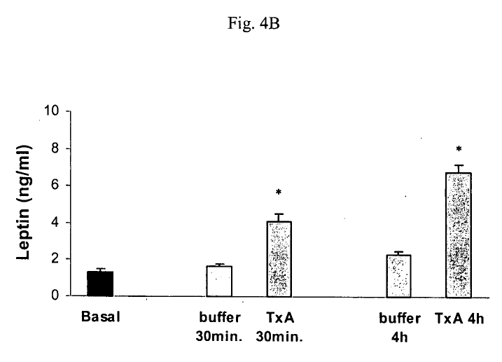

Mouse ileal loops: Twelve weeks old male C57BL/6J, ob/ob (B6.V-Lep^{ob}) and db/db (BKS.Cg-m+/+Lepr^{db}) mice (Jackson laboratory, Bar Harbor, Me.) weighing 20-25 g were housed on a 12-12 h light dark circle for 3 days prior to surgery. Experiments were performed between 9.30 am-11.30 am to minimize the influence of the circadian rhythm. Mice were fasted (16 hrs) and then anesthetized with a mixture of ketamine and xylazine. After laparotomy, two 3-5 cm ileal loops were formed and injected with 0.15 mL of phosphate buffer saline (PBS) (pH 7.4) containing 10 μg of purified TxA or buffer alone. Some ob/ob mice were administered i.p. with mouse recombinant leptin (1 μg/gr of body weight, Sigma, St. Louis, Mich.), 20, 14, and 0.5 hr prior to TxA administration. The abdomen was sutured, animals were placed on a heating pad at 37° C., and after 4 hr sacrificed with CO₂ inhalation. Fluid secretion was estimated as the loop weight to length ratio (mg/cm) as previously described (Castagliuolo, I. et al., 1998. J. Clin. Invest., 101:1547-50; Pothoulakis, C. et al., 1994. Proc. Natl. Acad. Sci. USA, 91:947-51). Full thickness loop sections were fixed in formalin, paraffin-embedded, stained with hematoxylin and eosin, and graded histopathologically using parameters associated with TxA-induced enterotoxicity. Data are depicted in FIGS. 4A and 4B.

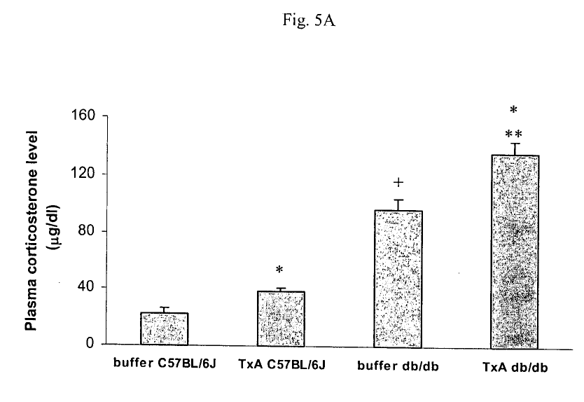

Adrenalectomy: Mice were adrenalectomized bilaterally under general anesthesia by the retroperitoneal route (Jacobson, L. et al., 1993. Neuroendocrinology, 58:420-9), while sham-operated mice underwent the same procedure without removing the adrenals. Adrenalectomized animals were given 0.9% NaCl to compensate for the salt loss. Six days following the adrenalectomy, ileal loops were formed, injected with TxA or buffer, and fluid secretion was measured after 4 hr. Plasma corticosterone before TxA and buffer injection was 1.30±0.16, and 1.60±0.25 μg/dL (mean±SEM) for adrenalectomized C57BL/6J (n=6) and db/db mice (n=6), respectively, compared to 22.0+3.8, and 85.0+5.4 μg/dL (mean±SEM) for non-adrenalectomized C57BL/6J, and db/db mice, respectively (n=4 for both groups). Animal studies were approved by the institutional animal care and use committee. Data are depicted in FIGS. 5A and 5B. Leptin and corticosterone measurements: Ileal loops of male wild-type mice were injected with 10 μg of purified TxA or buffer (control), and blood samples (0.2 mL) were collected from the retro-orbital plexus, centrifuged (800 g×10 min at 4° C.), and aliquots of plasma were stored at −80° C. Leptin and corticosterone levels were measured by a radioimmunoassay kits from Linco Research Institute (St. Louis, Mo.), and ICN Biomedicals, Inc. (Costa Mesa, Calif.), respectively.

TABLE 1
Reduced toxin A-mediated histologic responses in ob/ob and db/db mice

| | Epithelial | Congestion and | Neutrophil | MPO Units/gr |
|---|---|---|---|---|
| Treatment | Damage | edema | infiltration | protein |
| Buffer | 0.4 ± 0.24 | 0.5 ± 0.28 | 0.0 ± 0.0 | 29.16 ± 5.84 |
| C57BL/6J | | | | |
| TxA | 2.8 ± 0.16** | 2.5 ± 0.2** | 2.6 ± 0.2** | 108.2 ± 16.5* |
| C57NL/6J | | | | |
| Buffer db/db | 0.3 ± 0.2 | 0.1 ± 0.1 | 0.1 ± 0.1 | 30 ± 4.61 |
| TxA db/db | 1.16 ± 0.16*/++ | 1.14 ± 0.09**/++ | 1.0 ± 0.0*/++ | 51.3 ± 2.07*/+ |
| Buffer ob/ob | 0.46 ± 0.18 | 0.2 ± 0.13 | 0.4 ± 0.16 | 32.5 ± 3.57 |
| TxA ob/ob | 1.31 ± 0.11**/++ | 1.37 ± 0.18**/++ | 1.28 ± 0.18*/++ | 75.56 ± 3.4*/+ |
| Ileal loops were injected with 10 μg of toxin A (TxA) or PBS. After 4 hrs, animals were sacrificed, ileal loops were removed, and histologic severity of enteritis was graded by a score of 0-3 for epithelial cell damage, congestion and edema of the mucosa, and mucosal neutrophil infiltration. For MPO, ileal loops were removed and homogenized in ice-cold PBS containing a mixture of protease inhibitors. Homogenates were then centrifuged and aliquots from the supernatants taken for MPO measurements. | | | | |
| #Data are means ±SEM per group, n = 8-10 for histologic quantitation and 4 for MPO measurements, each with duplicate determinations. The non parametric Mann-Whitney test was used to calculate the histological differences. | | | | |

*, P < 0.05, and
**, P < 0.01 versus controls +,
++ P < 0.05, and P < 0.01 versus TxA of C57BL/6J mice.

TABLE 2
Increased toxin A-mediated Ob-Rb mRNA accumulation in mouse ileum.

| Time points | Treatment | Leptin mRNA |
|---|---|---|
| 1h | Buffer | 99.7 ± 6.6 |
| | TxA | 152.4 ± 20.4 |
| 2h | Buffer | 100.0 ± 6.8 |
| | TxA | 173.4 ± 15.8* |
| Ileal loops were injected with PBS or toxin A (TxA) and, after 1 and 2 hrs., RNA was extracted and reverse-transcribed to obtain cDNA. Ob-Rb mRNA was qualified by RT-PCR. Data represent the mean values (from arbitrary PhosphorImager units) if each group corrected for the β-actin values and then normalized to the mRNA expression of the buffer treated group, which is considered to be 100. Data are mean + SEM, n = 5 per group. Statistical significance was assessed by unpaired two-tailed | | |
| #Student's t-test using the Statview program (Abacus, CA). | | |

*denotes P < 0.001 vs buffer C57BK/6J mice.

Myeloperoxidase (MPO) measurements: MPO activity was determined by a modified method previously described (Bradley, P. et al., 1982. J. Invest. Dermatol., 78:206-209). Ileal loop samples were snap frozen, subjected to three rounds of freeze/thaw, and homogenized in 1 mL of 50 mM KH₂PO₄ buffer containing 0.167 mg/mL of O-dianisidine dihydrochloride and 5.10⁻⁴% of hydrogen peroxide. MPO activity was measured spectrophotometrically at 450 nm using human MPO (0.1 U/10 μL; Sigma) as a standard.

Total mucosal RNA extraction and Polymerase Chain Reaction (RT-PCR) amplification for Ob-Rb mRNA: Ileal loops were injected with either PBS or TxA. After 1 or 2 hr, the loops were removed, opened, washed in ice-cold PBS, and the mucosa was scraped with RNAse-free glass slides. Total RNA was isolated, and RT-PCR was performed as described previously (Bjorbaek, C. et al., 1999. Endocrinology, 140:2035-43). Briefly, a final volume of 100 μL cDNA was synthesized from 1 μg of total mRNA using the Advantage RT-PCR kit from Clontech (Palo Alto, Calif.). Ob-Rb cDNA was amplified using the following primers: Upstream: 5′-ACA GCG TGC TTC CTG GGT CTT C-3′ (SEQ. ID. NO: 1), Downstream: 5′-TGG ATA AAC CCT TGC TCT TCA-3′ (SEQ. ID. NO: 2). The 201 bp β-actin cDNA was amplified using the following primers: Upstream: 5′-CGT ACC ACG GGC ATT GTG ATG G-3′ (SEQ. ID. NO: 3), and Downstream: 5′-TTT GAT GTC ACG CAC GAT TTC CC-3′ (SEQ. ID. NO: 4). Preliminary PCR experiments showed that the rate of amplification was linear for β-actin when applied for fewer that 20 cycles and for Ob-Rb when applied for fewer than 35 cycles. Each 50 μL PCR reaction was carried out with 5.0 μL of template cDNA. Conditions were: 10 mM Tris-HCl (pH 8.8), 50 mM KCl, 1.5 mM MgCl₂, 0.01% gelatin, 0.2 mM dNTPs, 20 pmol of each primer, 2.5 U of Taq polymerase (Stratagene, La Jolla, Calif.), and 0.5 μL of ³²P-dGTP (29.6 Tbq/mmol, 370 Mbq/mL) (NEN, Boston, Mass.). The mixture was overlaid with 50 μL of mineral oil and, after initial denaturation at 96° C. for 4 min, the samples were subjected to 18 cycles of amplification for β-actin and to 30 cycles for Ob-Rb (denaturation at 95° C. for 1 min, annealing at 58° C. for 1 min, and extension at 72° C. for 45 sec). Ten μL of the reaction were then combined with 5 μL of sequencing stop solution (Amersham International, Buckinghamshire, UK) and heated to 85° C. for 5 min, before loading 4 μL onto a 4% urea-acrylamide gel (38×31×0.03 cm). Samples were electrophoresced at 60 W of constant power for 1.45 hr. After electrophoresis, gels were transferred to filter paper, dried and subjected to ³²p quantitation by PhosphoImager Analysis (Molecular Dynamics, Sunnyvale, Calif.).

Immunohistochemistry: TxA or buffer were injected into ileal loops of wild type mice (n=3 per group) and after 15, 30, and 60 min following TxA exposure or 60 min after buffer exposure, mice were killed and freshly frozen sections were prepared. Frozen ileal sections were cut (5 microns) and fixed in Streck tissue fixative for 10 min. Sections were then washed in Tris-buffered saline (pH 7.5) containing 0.1% Tween 20, (TBST, Dako Corporation), and incubated with 2% normal donkey serum in TBST for 30 min at 22° C. Sections were incubated for 2 hrs with a 1:20 dilution of rabbit anti-human Ob-Rb antibody or control rabbit IgG (Linco Research, Inc; St. Charles, Mo.), washed in 1×TBST and incubated for 30 min with a 1:50 dilution of a FITC-conjugated donkey anti-rabbit IgG (Jackson ImmunoResearch Laboratories, Inc., West Grove, Pa.). Sections were mounted with anti-bleaching solution and images viewed under a confocal microscope.

Statistical analyses: Unless otherwise stated, data was analyzed using the SIGMA-STATTM statistics software program (Jandel Scientific Software, San Rafael, Calif.). Analysis of variance with protected t test (ANOVA) was used for intergroup comparisons.

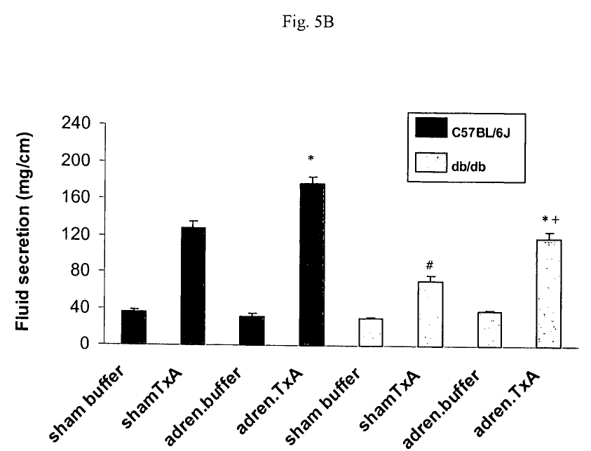

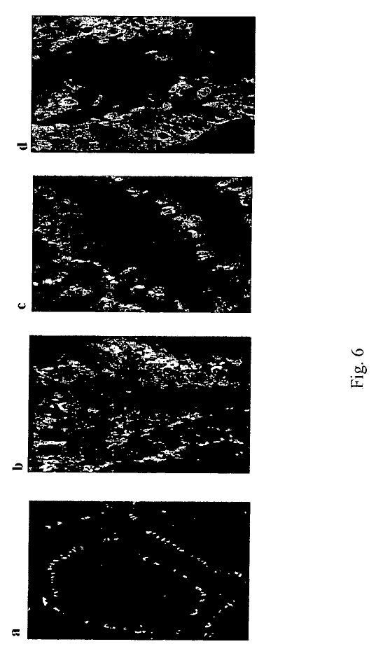

FIGS. 6A-D depict results of a representative experiment. Leptin receptor staining is present in control (buffer-exposed) mouse ileum. However, 15, 30 and 60 minutes after injection of TxA into ileal loops, there is a dramatic increase in the expression of leptin receptor compared to control. Note also that leptin receptor immunoreactivity is present on intestinal epithelial cells as well as in cells of the lamina propria. Taken together with the RT-PCR results, these data indicate a substantial upregulation of the leptin receptor in the early stages of TxA-induced enteritis.

All references, patents and patent applications cited are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method of inhibiting an inflammatory response in a tissue comprising leptin receptor positive cells, comprising administering to the tissue an agent that inhibits the signaling activity of the leptin receptor that mediates intestinal inflammation, thereby inhibiting the inflammatory response in the tissue.

2. The method of claim 1, wherein administering the agent comprises contacting the leptin receptor positive cells with the agent.

3. The method of claim 1, wherein the agent inhibits leptin receptor signaling, and wherein the agent is selected from the group consisting of: leptin receptor inhibitors, leptin derivatives, leptin analogs, anf antibodies that bind to the leptin receptor.

4. The method of claim 1, wherein the agent is a competitive inhibitor binding of leptin to the leptin receptor.

5. The method of claim 1, wherein the agent is a soluble isoform of the leptin receptor or a fragment thereof that retains the ability to bind to leptin.

6. The method of claim 1, wherein the agent inhibits binding to the leptin receptor, and wherein the agent is selected from the group consisting of: leptin antibodies, leptin receptor antagonists, leptin analogs and leptin derivatives.

7. The method of claim 6, wherein the leptin receptor antagonist is a peptide or peptide analog.

8. The method of claim 6, wherein the agent is an inhibitor of the leptin receptor binding activity of leptin.

9. The method of claim 1, where the inflammation is mediated by an autoimmune response, a parasite, a bacterium, a virus or a toxin.

10. The method of claim 9, where the toxin is produced by Clostridium difficile.

11. The method of claim 1, wherein the inflammation is of the small or large intestine.

12. A method for treating leptin-mediated intestinal inflammation in a mammal, comprising administering to a mammal an effective amount of an agent that inhibits the signaling activity of the leptin receptor that mediates intestinal inflammation, thereby inhibiting the inflammatory response in the tissue.

13. The method of claim 12, wherein administering the agent comprises contacting the leptin receptor positive cells with the agent.

14. The method of claim 12, wherein the agent inhibits leptin receptor signaling, and wherein the agent is selected from the group consisting of: leptin receptor inhibitors, leptin derivatives, leptin analogs, anf antibodies that bind to the leptin receptor.

15. The method of claim 12, wherein the agent is a competitive inhibitor binding of leptin to the leptin receptor.

16. The method of claim 12, wherein the agent is a soluble isoform of the leptin receptor or a fragment thereof that retains the ability to bind to leptin.

17. The method of claim 12, wherein the agent inhibits binding to the leptin receptor, and wherein the agent is selected from the group consisting of: leptin antibodies, leptin receptor antagonists, leptin analogs and leptin derivatives.

18. The method of claim 17, wherein the leptin receptor antagonist is a peptide or peptide analog.

19. The method of claim 17, wherein the agent is an inhibitor of the leptin receptor binding activity of leptin.

20. The method of claim 12, where the inflammation is mediated by an autoimmune response, a parasite, a bacterium, a virus or a toxin.

21. The method of claim 20, where the toxin is produced by Clostridium difficile.

22. The method of claim 12, wherein the inflammation is of the small or large intestine.

23. A composition for treating intestinal inflammation in a mammal, comprising one or more agents selected from the group consisting of: leptin antibodies, leptin agonists, leptin antagonists, non-biologically active leptin analogs, leptin receptor agonists or leptin receptor antagonists and a pharmaceutically acceptable carrier.

24. The composition of claim 23, wherein the agent inhibits leptin receptor signaling, and wherein the agent is selected from the group consisting of: leptin receptor inhibitors, leptin derivatives, leptin analogs, anf antibodies that bind to the leptin receptor.

25. The composition of claim 23, wherein the agent is a competitive inhibitor binding of leptin to the leptin receptor.

26. The composition of claim 23, wherein the agent is a soluble isoform of the leptin receptor or a fragment thereof that retains the ability to bind to leptin.

27. The composition of claim 1, wherein the agent inhibits binding to the leptin receptor, and wherein the agent is selected from the group consisting of: leptin antibodies, leptin receptor antagonists, leptin analogs and leptin derivatives.

28. The composition of claim 27, wherein the leptin receptor antagonist is a peptide or peptide analog.

29. The composition of claim 27, wherein the agent is an inhibitor of the leptin receptor binding activity of leptin.

30. Use of an inhibitor of leptin or a leptin receptor for the manufacture of a medicament for the treatment of an inflammatory disease or condition.

31. The method of claim 1, claim 12, claim 23 or claim 30, where the inflammation is associated with an autoimmune response, a parasitic infection, inflammatory bowel disease, Crohn's disease, ulcerative colitis, acute enterocolitis or chronic enterocolitis.


SEQUENCE LISTING (4 sequences)

SEQ ID NO: 1 - DNA, 22 bp, Artificial Sequence
  PCR Primer
acagcgtgcttcctgggtcttc

SEQ ID NO: 2 - DNA, 21 bp, Artificial Sequence
  PCR Primer
tggataaacccttgctcttca

SEQ ID NO: 3 - DNA, 22 bp, Artificial Sequence
  PCR Primer
cgtaccacgggcattgtgatgg

SEQ ID NO: 4 - DNA, 23 bp, Artificial Sequence
  PCR Primer
tttgatgtcacgcacgatttccc